(19) United States
(12) Patent Application Publication
(10) Pub. No.: US 2026/0240430 A1
(43) Pub. Date: Aug. 20, 2026

(54) VISUAL-ACUITY-RELATED ENHANCEMENT FOR IMPROVED CONTENT PRESENTATION ON HEAD-MOUNTED DEVICES

(71) Applicant: Heru Inc., Miami, FL (US)

(72) Inventors: Mohamed ABOU SHOUSHA, Fort Lauderdale, FL (US); Alexander MILLER, Miami, FL (US); Rashed KASHEM, Miami, FL (US); Terry Keith BROCK, Miami, FL (US)

(73) Assignee: Heru Inc., Miami, FL (US)

(21) Appl. No.: 19/057,965

(22) Filed: Feb. 19, 2025

(51) Int. Cl. A61B 3/04; A61B 3/032; G06F 3/01; G06F 3/0346; G06F 3/0362; G06T 7/73; G06T 15/10

(52) U.S. Cl. CPC A61B 3/04; A61B 3/032; G06F 3/013; G06F 3/0346; G06F 3/0362; G06T 7/73; G06T 15/10; A61B 2560/0238; G06T 2207/30041; G06T 2210/41

(57) ABSTRACT

A head-mounted device and related method may increase visual acuity during content presentation by determining spatial displacements between an eye and the lens vertex and applying transformation parameters to ensure accurate visual alignment during visual acuity testing.

### SUMMARY

Visual acuity measures how well eyes can distinguish fine details at specific distances, affecting daily tasks and overall function. Testing visual acuity enables early detection of vision problems and guides corrective lens prescriptions. Regular assessment helps monitor eye health and track changes in vision over time. Visual acuity testing uses standardized symbols presented at specific sizes and distances to measure the eye's ability to resolve details. However, conventional visual acuity testing issues include inconsistent testing distances, variable room illumination, and patient fatigue affecting the reliability of measurements.

Head-mounted devices may help address such issues by controlling for illumination, providing a standardized virtual environment for testing, and reducing testing time to reduce the likelihood of patient fatigue. However, the use of head-mounted devices presents unique challenges. One such challenge is that a head-mounted device may force a viewer to view image content through a lens. This setup can lead to misalignments due to spatial displacement between the physical center of the pupil and the vertex (center) of the lens. When such misalignments occur, the eye is not looking directly through the lens vertex, which is the optimal point for clear vision. As a result, such misalignments can artificially reduce a maximum visual acuity because the eye is viewing the content through a less optimal part of the lens.

Some embodiments may resolve such issues and other issues by determining displacements between an eye and a lens vertex and using this information to transform displayed image content so that the eye views such content through the lens vertex. Some embodiments may present a first stimulus at a first display position on a head-mounted display of a head-mounted device and then determine, via one or more sensors, the pupil center of a pupil directed at the first stimulus. For example, some embodiments may present a first stimulus at a first position [0, 0, 0] on a head-mounted display of a head-mounted device and then use one or more inward-facing infrared sensors to determine the spatial position of a pupil directed at the first stimulus. Some embodiments may determine a spatial displacement between the pupil center and a vertex of a headset lens of the head-mounted display and generate transformation parameters based on the spatial displacement. For example, some embodiments may determine a spatial displacement between a pupil center determined to be at [−1, −1, −15] in physical space with respect to a vertex that is set at the origin [0, 0, 0] for the physical space. Based on this spatial displacement, some embodiments may generate transformation parameters by applying trigonometric functions, sophisticated rules-based models, statistical models, or machine learning models. Some embodiments may then determine a second display position by applying a transformation to an initial presentation position based on the transformation parameters and present the second stimulus at the second display position.

By performing operations and using related devices described in this disclosure, some embodiments may enhance the reliability of visual acuity testing by using head-mounted devices to create a controlled virtual environment. Furthermore, the operations described in this disclosure address issues like inconsistent testing distances and variable room illumination. Additionally, the efficient manner or even passive manner of data collection may reduce patient fatigue by shortening the testing time, leading to more accurate measurements. Furthermore, the operations described in this disclosure may help mitigate the deleterious effects of misalignments between a pupil and a lens vertex by increasing the likelihood that the eye views content through the lens vertex.

Various other aspects, features, and advantages of the invention will be apparent through the detailed description of the invention and the drawings attached hereto. It is also to be understood that both the foregoing general description and the following detailed description are examples and are not restrictive of the scope of the invention.

### BRIEF DESCRIPTION OF THE DRAWINGS

FIG. 1 shows an example system for increasing visual acuity during operations of a head-mounted device, in accordance with one or more embodiments.

FIG. 2 shows an example head-mounted device, in accordance with one or more embodiments.

FIG. 3 shows an example conceptual architecture for increasing visual acuity during operations of a head-mounted device, in accordance with one or more embodiments.

FIG. 4 shows a flowchart of a process for increasing visual acuity during operations of a head-mounted device, in accordance with one or more embodiments.

The technologies described herein will become more apparent to those skilled in the art by studying the detailed description in conjunction with the drawings. Embodiments of implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION OF THE DRAWINGS

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

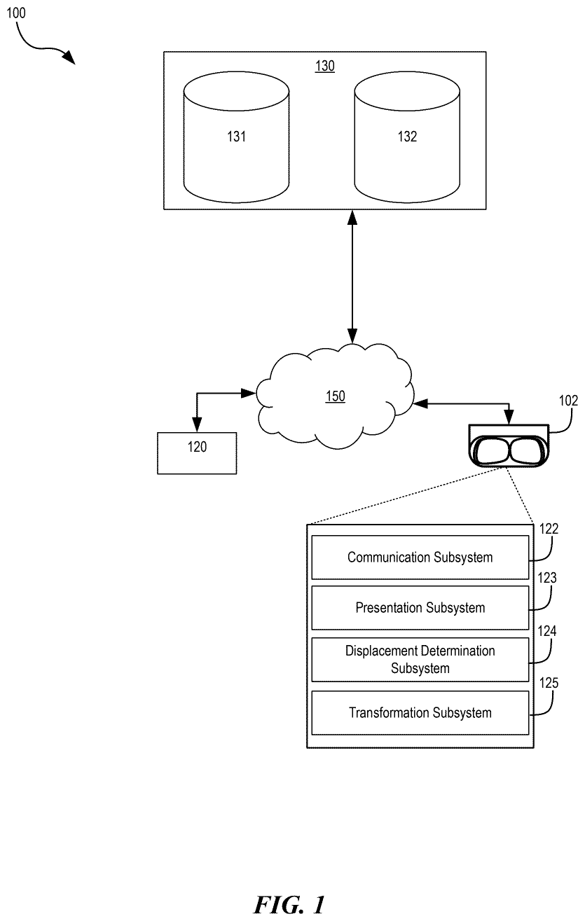

FIG. 1 shows an example system 100 increasing visual acuity during operations of a head-mounted device, in accordance with one or more embodiments. A system 100 includes a client device 102 in communication with a server 120 via a network 150. As will be described further in this disclosure, the client device 102 may perform operations to increase visual acuity of a presented object by transforming display positions of the object such that an eye's gaze is directed to the object through a lens vertex.

In some embodiments, the system 100 may increase accuracy of visual acuity testing or other presentations of content by determining displacements between an eye and a lens vertex. Some embodiments. Some embodiments may initialize correction operations when starting a visual acuity test for a user and present a calibration stimulus at an optical infinity position or another calibration display position on the head-mounted display. When the calibration stimulus is presented, a user may consciously or subconsciously use their eye to focus on the calibration stimulus. By presenting a stimulus at a known calibration position, some embodiments may be able to establish a baseline position for eye orientation, where data collected by sensors may be used to determine displacements. Some embodiments may then determine, with one or more sensors, the pupil center of a pupil directed at the calibration stimulus while in a primary gaze position, and then determine a spatial displacement between the pupil center and a vertex of the headset lens. Additionally, some embodiments may then generate transformation parameters based on the spatial displacement and present a testing stimulus, stimulus in video content, stimulus in gaming content, or other types of content during a visual acuity test by applying the transformation to adjust the stimulus position and display it accurately on the head-mounted display.

Some embodiments may start correction operations for a visual acuity test by presenting a calibration stimulus at a calibration position on the head-mounted display. Some embodiments may then determine a pupil center of a pupil directed at the calibration stimulus and a spatial displacement between the pupil center and a vertex of a headset lens of the head-mounted device. For example, some embodiments may then determine, via one or more inward-facing infrared sensors, the pupil center of a pupil directed at the calibration stimulus and an associated spatial displacement value indicating a distance and direction to a lens vertex. By determining this spatial displacement, some embodiments can correct for any viewing misalignments between an eye and a lens caused by spatial difference between the eye's position and the lens' vertex. Some embodiments may then generate transformation parameters based on the spatial displacement and present a stimulus by applying the transformation to adjust the stimulus position and display it accurately on the head-mounted display.

Some embodiments may present a calibration stimulus at a first display position on a head-mounted display, determine the pupil center of a pupil directed at the calibration stimulus via one or more sensors, and then determine the spatial displacement between the pupil center and a vertex of a headset lens. Some embodiments may then generate a set of transformation parameters based on the spatial displacement and apply a transformation to an initial position for a new stimulus using these parameters to determine an adjusted position on the head-mounted display. Some embodiments may compute the set of transformation parameters to correct for any misalignments between the pupil center and the lens vertex in a head-mounted display. By determining the spatial displacement and generating these parameters, the system can apply necessary transformations to the displayed content, ensuring that the eye views the content through the lens vertex. These transformations may enhance the accuracy and reliability of visual acuity testing by correcting for error-inducing misalignments that would result in a line of sight passing through a lens without passing through the vertex. Since a visual acuity test might often presume that a patient is seeing through the clearest portion of a lens (e.g., the vertex), such a misalignment could artificially reduce a perceived or measured visual acuity. Similarly, visual acuity would in general be reduced by such a misalignment during the presentation of other types of content, such as gaming content or video content. Thus, such transformations may mitigate the artificial reduction in visual acuity in head-mounted devices. After determining the transformation parameters or applying transformations based on the transformation parameters to determine adjusted display positions, some embodiments may then display content based on the adjusted display positions. For example, some embodiments may display one or more optotypes or other types of stimuli at the adjusted display positions on the head-mounted device.

The client device 102 may include a head-mounted device, such as a virtual reality device or an augmented glasses device. The client device 102 may send requests, responses, or other messages to the server 120 that may require communication with other computing devices or other electronic devices. Additionally, the server 120 may include various types of computing units, such as physically separate servers, virtual nodes hosted on one or more physical machines, or nodes on a cloud computing system. Applications, services, or other operations may use data provided by the client device 102, the server 120, or a set of databases 130 that includes a first networked database 131 and a second networked database 132. The set of databases 130 may include various types of databases, such as SQL databases, no SQL databases, graph databases, etc. In some embodiments, the client device 102 may perform one or more operations related to a communication subsystem 122, a presentation subsystem 123, a displacement determination subsystem 124, or a transformation subsystem 125. The server 120 may perform operations such as training one or more machine learning operations described in this disclosure based on feedback data, generating machine learning models, storing feedback data, storing historical feedback data, etc.

In some embodiments, the communication subsystem 122 may obtain program instructions, commands, parameters, values, or other data from the server 120 or the set of databases 130. For example, the communication subsystem 122 may retrieve a set of parameters from the set of databases 130. Furthermore, operations performed by the client device 102 may use the communication subsystem 122 to send messages to the set of databases 130, the server 120, or another computing device described in this disclosure. Furthermore, some embodiments may use the communication subsystem 122 to communicate with one or more remote computing devices to offload some or all of the operations described in this disclosure. For example, some embodiments may use the communication subsystem 122 to provide the server 120 with measurements of eye positions or displacements obtained from sensors of the client device 102. The server 120 may then determine transformation parameters based on the measurements, and then send the transformation parameters back to the client device 102. Alternatively, or additionally, some embodiments may transform image data before providing the transformed image data to the client device 102 for presentation.

In some embodiments, the presentation subsystem 123 may present image content to a user that is viewed by the user's eye. For example, some embodiments may use the presentation subsystem 123 to present a first stimulus at a first display position on a head-mounted display of a head-mounted device, such as by presenting a calibration stimulus at an optical infinity position. Such operations allow a user's eyes to focus on a distant point to determine an angle at which an eye is in a primary gaze position. Furthermore, some embodiments may, as a part of a visual acuity, present a second stimulus at a second display position by applying a transformation to an initial position associated with the second stimulus based on the transformation parameters. For example, some embodiments may apply a translational transformation that causes a −0.5 shift in the horizontal direction, a +1 shift in the vertical direction, and a no shift in the depth direction. After applying this translational transformation to an initial position [0, 0, −10], some embodiments may obtain an adjusted display position [−0.5, 1, −10]. Furthermore, may use the presentation subsystem 123 to perform additional visual testing operations, such as operations for a contrast sensitivity test or a subjective refraction test.

In some embodiments, the presentation subsystem 123 may present various other types of stimuli in image content that is to be viewed by the user's eye. As used in this disclosure, stimulus may include any type of content that either can elicit a reaction in an eye or is designed to elicit a reaction in an eye. For example, a stimulus may include an object that contrast with a surrounding environment with respect to color or brightness, an object that is animated, an object that is instantiated in a viewer's visual field, etc. Various types of content may introduce stimuli, including testing applications, video-presenting applications, videogame applications, or other types of applications. For example, the presentation subsystem 123 may present stimulus used in gaming content, such as a ball being shown at a first display position on a head-mounted display of a head-mounted device. Furthermore, as described elsewhere in this disclosure, some embodiments may apply a transformation to an initial position associated with the vertices representing the ball based on transformation parameters to correct for a misalignment and improve visual acuity.

In some embodiments, the displacement determination subsystem 124 may obtain eye-related measurements and determine displacements from these measurements. Some embodiments may determine a physical pupil position of a pupil directed at a first stimulus. For example, inward-facing sensors may track the user's eye movements as the user looks at a calibration stimulus to determine an angle away from a primary gaze position. Some embodiments may use these measurements to calculate a spatial displacement between a detected pupil center position and a vertex of a lens of the client device 102. For example, the displacement determination subsystem 124 may determine a physical pupil center position in a two-dimensional or three-dimensional space and then determine a displacement from the vertex of the client device 102.

In some embodiments, the transformation subsystem 125 may use a spatial displacement between a detected pupil center position or another eye-related position and the vertex of the headset lens to generate transformation parameters. These transformation parameters may be used for applying a translation transformation, rotation transformation, or warping transformation. For example, the system may use calculated horizontal, vertical, and depth displacement of an eye as inputs for a set of trigonometric functions or more sophisticated functions to generate a translation matrix and a rotation matrix. Some embodiments may then combine the translation matrix and a rotation matrix (e.g., by multiplying them) to determine a transformation matrix that is applied to a stimulus. The transformation subsystem may then use the transformation matrix to adjust the display content adapted to a user's eye position and improve visual accuracy. For example, if the user's eyes have shifted slightly to the left, the transformation subsystem applies the transformation parameters to shift an optotype or another stimulus, accordingly, in a rightward fashion, ensuring that the user's vision passes through the vertex to view the optotype or the other stimulus.

Some embodiments may apply a transformation based on determined transformation parameters to various types of applications, such as vision testing, gaming, simulated environmental exploration, or video content presentation. For example, in the context of gaming, the transformation subsystem 125 may apply a translational transformation that causes a shift in the horizontal and vertical directions in the visual presentation of a ball that is being thrown at a user in the virtual environment of a head-mounted device. Alternatively, in the context of presenting a movie or other video content, the transformation subsystem 125 may apply a translational transformation to modify the viewing window of the video content such that the center of the video content being presented is shifted or rotated by the transformation subsystem 125. For example, if the initial position of the center of the video content is at [0, 0, −100] in virtual space, the transformation subsystem might apply a −10 shift in the horizontal direction and a +10 shift in the vertical direction, resulting in an adjusted display position of [−10, 10, −100] in a virtual space being presented by a head-mounted device.

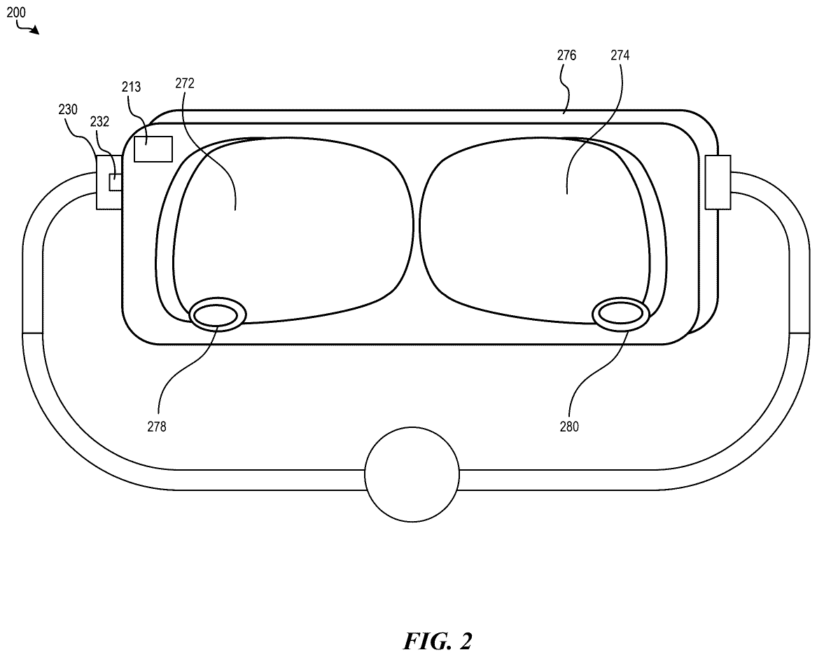

FIG. 2 shows an example head-mounted device 200, in accordance with one or more embodiments. A head-mounted device 200 may be used as a content presentation device that may concurrently detect and analyze vision systems. This head-mounted device 200 may include two components: a left eyepiece 272 and a right eyepiece 274. Both eyepieces 272 and 274 are equipped with digital monitors that can either display or project reconstructed images directly to the wearer's eyes.

The digital monitoring system is housed within a frame structure 276, with the left eyepiece 272 and right eyepiece 274 carefully positioned to align with the wearer's orbital area. This arrangement enables the left eyepiece 272 and the right eyepiece 274 to both gather information and present visual data, with each component serving a distinct eye. The frame structure 276 also includes a depth-controlling slider 230 that includes a position sensor 232. The position sensor 232 may determine an objective or subjective depth measurement of the frame structure 276 or a component of the head-mounted device 200 (e.g., the left eyepiece 272 or the right eyepiece 274) is from a user.

The system features a left inward-facing sensor 278 and a right inward-facing sensor 280. For example, the left inward-facing sensor 278 or the right inward-facing sensor 280 may utilize infrared technology for cornea tracking or other types of eye tracking activity. The left inward-facing sensor 278 and the right inward-facing sensor 280 may be placed in the lower sections of the left eyepiece 272 and the right eyepiece 274, respectively. Such positioning of the left inward-facing sensor 278 and the right inward-facing sensor 280 may avoid interfering with a user's field of vision. In some embodiments, the left inward-facing sensor 278 and the right inward-facing sensor 280 may be specifically oriented toward the expected pupil position to optimize cornea tracking capabilities or other types of eye tracking capabilities. Furthermore, in some embodiments, the left inward-facing sensor 278 and the right inward-facing sensor 280 may be embedded within the left eyepiece 272 and the right eyepiece 274, respectively, to create a smooth interior surface.

In some embodiments, a set of processors 213 of the head-mounted device 200 may execute one or more operations described in this disclosure, such as an operation to determine displacements, transformation parameters, apply transformations, etc. Some embodiments may use data provided by the left inward-facing sensor 278 to determine displacement information for the left eye and use data provided by the right inward-facing sensor 280 to determine displacement information for the right eye. The set of processors 213 may then determine a corresponding set of translation transformation parameters or rotation transformation parameters for the left and right eyes using one or more operations described in this disclosure. The set of processors 213 may further apply transformations to the images being displayed via the left eyepiece 272 and the right eyepiece 274 based on these transformation parameters.

It should be understood that transformation operations described in this disclosure may be applied to various types of applications, including but not limited to vision testing application, videogaming applications, etc. For example, the set of processors 213 may execute an application to present a simulated three-dimensional environment via the left eyepiece 272 and the right eyepiece 274. The set of processors 213 may then operations to measure displacements, calculate transformation parameters, and apply transformations based on the transformation parameters to the visual presentation of the simulated three-dimensional environment. For example, some embodiments may present various stimuli such as virtual trees, animals, and rocks as a user is exploring a virtual forest in a simulated environment. Some embodiments may then determine an eye-to-vertex distance based on a viewer's focus on a first stimulus, (e.g., a red fruit at a known display position on a display screen) and determine transformation parameters based on the distance. Some embodiments may then apply a transformation based on these transformation parameters to modify the presentation of the virtual forest. For example, some embodiments may apply an additional filter is applied to image content to be presented to a user, where the additional filter may shift the presentation in a certain direction or rotate the presentation in a certain orientation.

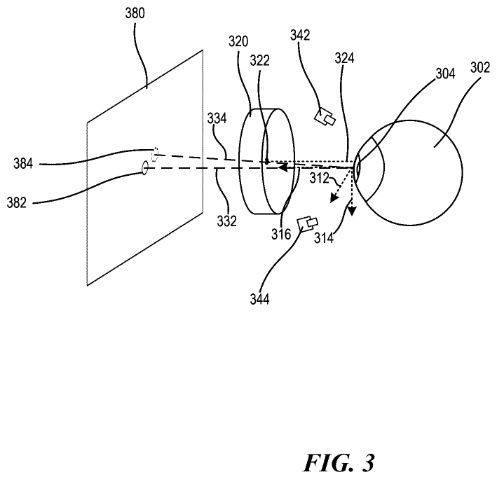

FIG. 3 shows an example conceptual architecture for increasing visual acuity during operations of a head-mounted device, in accordance with one or more embodiments. A head-mounted display 380 first presents a calibration stimulus 382. As shown by the sight line 332, while the sight line 332 does pass through a lens 320 that is between the eye 302 and the head-mounted display 380, the eye 302 does not look directly through a vertex 322 of the lens 320. Also, a normal lens center line 324 may be normal with respect to the surface of the lens 320 and intersect the vertex 322.

In some embodiments, a first inward-facing sensor 342 and a second inward-facing sensor 344 may observe the eye 302. This observation may reveal the orientation and physical position of parts of the eye 302, including a pupil 304 of the eye 302. The feedback data provided by the first inward-facing sensor 342 and the second inward-facing sensor 344 may be used to determine a horizontal displacement 312, a vertical displacement 314, and a depth displacement 316. As shown by the arrow for the horizontal displacement 312, the physical center position of the pupil 304 is slightly to the left of the vertex 322. As shown by the arrow for the vertical displacement 314, the physical center position of the pupil 304 is slightly below the vertical position of the vertex 322. Some embodiments may use this information to generate a translational transformation matrix or another collection of transformation parameters usable to modify additional stimuli or other objects to be displayed on the head-mounted display 380.

Various types of transformation operations may be applied to transform objects displayed on the head-mounted display 380. As a first example, some embodiments may directly reverse a horizontal displacement and vertical displacement. For example, based on the value of horizontal displacement 312 and the value of vertical displacement 314, some embodiments may modify a future display of a testing stimulus 384. For example, when presented at a same distance from the vertex 322 in virtual space as that of the depth displacement 316, some embodiments may shift the testing stimulus 384 rightward by the magnitude of the horizontal displacement 312 and shift the testing stimulus 384 upward by the magnitude of the vertical displacement 314. Alternatively, or additionally, some embodiments may apply a set of trigonometric functions to determine a transformation matrix usable for achieving this effect. For example, if the angle formed by the normal lens center line 324 and the viewing direction of the eye 302 forms a first angle θ in the horizontal direction and forms a second angle φ in the vertical direction, and if the depth displacement 316 has a length equal to d, some embodiments may visually shift a stimulus that would have been presented at the coordinates [0, 0] (representing horizontal and vertical directions on a display) at the coordinates [d sin(θ), d sin(φ)]. It should be understood that additional or alternative transformation operations may be performed to account non-linear behaviors in an eye, eye defects, lens defects, etc. Furthermore, it should be understood that various other transformations may be applied that are specific to an application or function.

Some embodiments may then apply a transformation operation to coordinates for the testing stimulus 384 using transformation parameters determined from the horizontal displacement 312, the vertical displacement 314, and the depth displacement 316. After the application of the transformation operation, some embodiments may present the testing stimulus 384 at the updated coordinates, where the pupil center of the pupil 304 may then form a direct line of sight to the testing stimulus 384 through the vertex 322, as shown by a sight line 334. In contrast, if the transformation operation had not been applied, some embodiments may have presented the testing stimulus 384 at the same position as that of the calibration stimulus 382.

### Flowchart

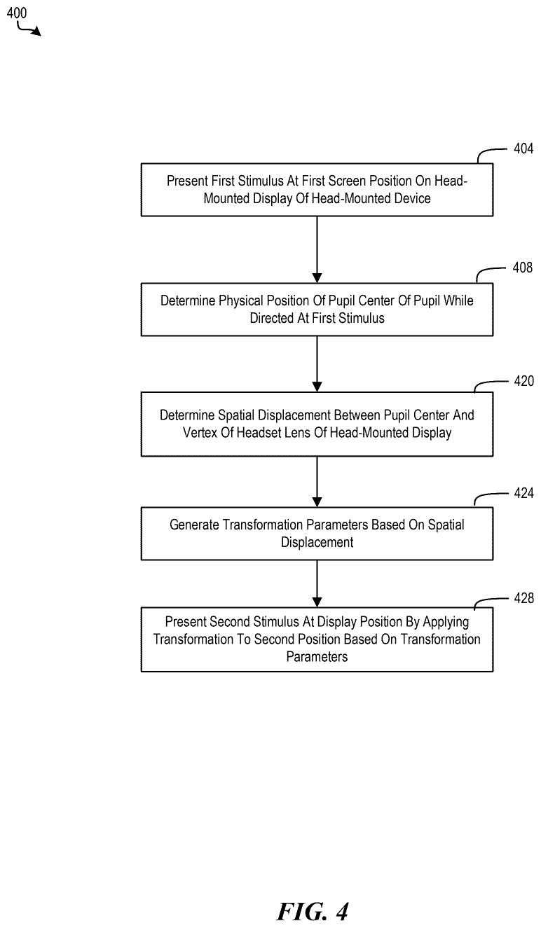

FIG. 4 shows a flowchart of a process 400 for increasing visual acuity during operations of a head-mounted device, in accordance with one or more embodiments. Some embodiments may present a first stimulus at a first display position on a head-mounted display of a head-mounted device, as described in block 404. Some embodiments may present a calibration stimulus at an optical infinity position on the head-mounted display. For example, while a user is watching a video on a head-mounted device, the device presents a calibration stimulus at an optical infinity position. This allows the user's eyes to focus on a distant point, simulating a real-world scenario where the eyes are relaxed and in a primary gaze position.

Some embodiments may select a calibration position that is at optical infinity position, where the optical infinity position may include but not be limited to [0,0] for the vertical and horizontal positions. Alternatively, some embodiments may select a calibration position other than an optical infinity position. For example, some embodiments may select, as a calibration position, a display position of a known stimulus during a device start-up process or an application start-up process that is not set at an origin position with respect to horizontal and vertical positions. Such a stimulus may include a logo, flashing object, an animated object, etc.

Alternatively, or additionally, some embodiments may initialize calibrations operations in the middle of an application. For example, some embodiments may determine an orientation shift between a head and a head-mounted device. The orientation shift may indicate the degree by which a head inside of the head-mounted device has shifted while the head-mounted device is being worn and, in response, begin or restart a calibration operation to improve visual acuity by updating transformation parameters. For example, some embodiments may use optical sensors, infrared sensors, or depth sensors to determine an orientation shift indicating a change in position of a user's eyes, face, or another part of the user's head relative to a head-mounted device. Some embodiments may then determine that the orientation shift is greater than a shift threshold and, in response, initiate a calibration operation. In some embodiments, the initiation of the calibration operation may be performed without the knowledge of a user. For example, some embodiments may passively wait until an appropriate stimulus is presented and measure a user's eye response using an inward-facing sensor to determine displacement values and update a set of transformation parameters.

Some embodiments may determine a physical pupil position of a pupil directed at the first stimulus, as described in block 408. Some embodiments may determine, with the inward-facing sensor, a physical pupil center position of a pupil directed at the calibration stimulus, wherein the pupil is in a primary gaze position while directed at the calibration stimulus. For example, some embodiments may use inward-facing sensors to track the user's eye movements and, while the user looks at a calibration stimulus, the sensors may determine the physical pupil center position. Alternatively, some embodiments may passively detect display data being projected on a head-mounted device to detect when a stimulus is displayed at a known display position. Some embodiments may then determine a physical position of the pupil center based on the known display position.

Some embodiments may determine a spatial displacement between the physical pupil position and a vertex of a headset lens of the head-mounted display, as described in block 420. Some embodiments may determine a spatial displacement between the physical pupil center position and a vertex of the headset lens. Some embodiments may calculate the spatial displacement between the detected pupil center position and the vertex of the headset lens. For example, inward-facing sensors may track a user's eye movements as the user is presented with a calibration stimulus at an optical infinity position on a display. The inward-facing sensors may then detect the physical pupil center position of the user's eyes as they focus on the calibration stimulus. For example, some embodiments may use infrared illuminators that shine infrared light onto the eyes and use infrared cameras that capture the reflected images. Some embodiments may then identify features such as the pupil, a corneal reflection (“glint”), an iris pattern, etc. In some embodiments, the use of multiple infrared illuminators to create several glints permits some embodiments to measure positions relative to the pupil center and determine a physical pupil center position in two-dimensional space or three-dimensional space.

In some embodiments, a lens vertex may be calculated based on a physical set of properties associated with the lens. Alternatively, some embodiments may store data that indicates a lens vertex position. For example, a head-mounted device may include a Fresnel lens between a user's eyes and a display screen. During a calibration operation or another operation described in this disclosure, some embodiments may retrieve a position representing the vertex from a data store based on the device type identifier. For example, some embodiments may set a lens vertex as a physical origin position [0, 0, 0] and, using a set of inward-facing sensors, determine that a physical pupil center position is at [1, −2, 12].

When determining an eye-related physical position such as a physical pupil center position, some embodiments may detect an angle of eye orientation and determine a corresponding horizontal displacement or a vertical displacement from a normal direction of a lens at a vertex. Some embodiments may retrieve a depth that an eye is from a lens vertex and apply trigonometric functions (e.g., sin functions applied to measured angles of an eye) to determine a horizontal displacement and a vertical displacement based on a measured angle of the eye. As a simplified example, some embodiments may first retrieve an estimated depth displacement from a configuration file. Some embodiments may then generate a first transformation parameter equal to d*Sin(phi) and a second transformation parameter equal to d*Sin(theta), where d is an estimated depth displacement, and where phi and theta may be the measured horizontal and vertical angles. Some embodiments may then generate a translation matrix based on a set of translation parameters that includes the estimated depth displacement, the first transformation parameter, and the second transformation parameter.

Some embodiments may obtain, from a data store, a parameter indicating a depth parameter, where this obtained parameter may then be used as a transformation parameter or be used to generate transformation parameters. For example, some embodiments may retrieve the value “30” to represent the value “15 mm” from a data store, where the value may range between 8 mm and 25 mm. This value may represent an average distance between an eye and a headset lens. Some embodiments may store multiple possible depth displacements and retrieve one or more depth displacements based on a specific device model. For example, some embodiments may determine that a user is wearing a device model “Type 1” and, in response, retrieve a displacement value “12 mm.” In a different device, some embodiments may determine that a user is wearing a device model “Type 2” and, in response, retrieve a displacement value “20 mm.”

Alternatively, some embodiments may use a depth-controlling slider to determine a distance to an eye. Some embodiments may determine a depth parameter using measurements from a position sensor that is attached to a depth-controlling slider of the head-mounted device. For example, in some embodiments, a head-mounted device may be equipped with a depth-controlling slider that allows the user to adjust the distance between the display and their eye. A position sensor may be attached to this depth-controlling slider to measure the exact position of the slider. Some embodiments may determine a depth parameter based on the measurements provided by this position sensor, such as adding to or subtracting from a reference depth.

Alternatively, some embodiments may use multiple infrared illuminators or multiple sensors to more accurately detect the physical distance an eye is from a lens vertex based on the measurements of the position of multiple infrared glints. For example, some embodiments may use two glints by first determining angles with respect to illuminators to a retina, determine a distance between the glints, and then determine a depth value based on the glint distances. Some embodiments may use a function or mapping between inter-glint distances and an estimated distance an eye is from another component of a head-mounted device, such as a lens vertex. For example, some embodiments may predict the pupil-to-vertex distance is 15 ml based on a determination that a glint distance is equal to “4.93 mm” by accessing a data store storing a conversion table and some embodiments may predict the pupil-to-vertex distance is 20 ml based on a determination that a glint distance is equal to “3.7 mm” by accessing the same data store. Some embodiments may use additional illuminators or sensors to determine an eye position.

Some embodiments may generate transformation parameters based on the spatial displacement, as described in block 424. Based on spatial displacement data, some embodiments may generate transformation parameters for applying a translation transformation, rotation transformation, warping transformation, or other types of transformations. Some embodiments may generate a transformation matrix by combining a translation matrix, a rotation matrix, or other matrices. Some embodiments may use the transformation matrix to adjust the display content to match the user's eye position and improve visual accuracy.

Some embodiments may further correct for rotations by determining one or more rotation parameters, where such operations may include providing multiple stimuli to a user and measuring the eye response as the multiple stimuli are being presented. For example, after presenting a first calibration stimulus at an optical infinity position [0, 0] for a head-mounted display, some embodiments may present a second calibration stimulus at the second position [1, 0] for the head-mounted display. Some embodiments may use a set of inward-facing infrared sensors or other sensors to determine a first pupil orientation when a pupil is directed at the first calibration stimulus and a second pupil orientation when a pupil is directed at the second calibration stimulus. Some embodiments may then determine a rotation angle indicating the angle based on the direction that the pupil moves as it moves from the first calibration stimulus to the second calibration stimulus. Some embodiments may then generate a rotation parameter usable for applying a rotation transformation based on the rotation angle. Some embodiments may then use the rotation parameter as part of a transformation matrix or otherwise update a transformation matrix based on the rotation parameter. For example, some embodiments may generate a rotation matrix based on a rotation angle determined from using a set of eye-related measurements indicating spatial displacements. Some embodiments may then generate a translation matrix based on the spatial displacements and then apply matrix multiplication with the rotation matrix to determine a final transformation matrix that includes transformation parameters. When applying a transformation with a set of transformation parameters, some embodiments may then multiply the coordinates of one or more stimuli to be presented by the final transformation matrix.

Some embodiments may use a machine learning model to augment or replace other transformation operations described in this disclosure when determining a set of transformation parameters. For example, some embodiments may obtain sensor data indicating the spatial displacement between a user's eyes and a head-mounted display. Some embodiments may then provide this spatial displacement data to a machine learning model stored on the local memory of the head-mounted device. Some embodiments may use a machine learning model based on an ability to dynamically adjust display content in real-time. For example, some embodiments may use a linear regression-based model or a shallow neural network model. Some embodiments may be configured to use a linear regression-based model based on the relative speed and performance of such models relative to other models. Some embodiments may favor the use of a neural network model due to an improved ability to account for small nonlinearities in the relationship between eye-related measurements and the optimal transformation parameters to be applied to an image.

Some embodiments may present a second stimulus at a display position by applying a transformation to a second position based on the transformation parameters, as described in block 428. Some embodiments may present a testing stimulus as the second stimulus. Some embodiments may present the testing stimulus to the user during a visual acuity test while compensating for the spatial displacement by applying a translational transformation to a testing stimulus position based on the translational transformation parameters. For example, some embodiments may determine an adjusted testing position by applying matrix multiplication operations to multiply a transformation matrix with a vector representing an initial presentation position associated with a new stimulus. The result of the multiplication operation or another transformation operation may be or may include an adjusted testing stimulus position. Some embodiments may then present the testing stimulus at the adjusted testing stimulus position on the head-mounted display. For example, some embodiments may have originally received instructions to present a red dot being used as a testing stimulus at the [0, 0, 0] position, applied a transformation to this position based on a set of translational transformation parameters to obtain an adjusted testing stimulus position equal to [1, −1, 0], and then present the red dot at the [1, −1, 0].

Some embodiments may perform other types of tests in addition to, or in lieu of, a visual acuity test. Some embodiments may perform a contrast sensitivity test (e.g., displaying a grating pattern that includes at least one lighter color and at least one darker color) at the test position after calibration. Visual acuity impairments can make it challenging to perform contrast tests reliably, as patients need to be able to see the basic elements of the test patterns to evaluate their contrast differences. For example, some embodiments may first determine a set of transformation parameters using the operations described in this disclosure. Some embodiments may then transform a grating pattern or another object that includes a lighter color and a darker color at testing position determined from applying the set of transformation parameters.

Some embodiments may perform a subjective refraction test at the test position after calibration. For example, after performing a calibration test to determine a testing display position for a display, some embodiments may display an optotype or some other type of stimulus at the testing display position for a subjective refraction test. During the subjective refraction test, some embodiments may present the stimulus at the testing position on the head-mounted display. As a part of the refraction test, some embodiments may then obtain a first feedback parameter associated with the optotype or another additional stimulus. In some embodiments, the first feedback parameter may indicate a relative clarity of the optotype or the additional stimulus. Based on this feedback, some embodiments may then modify a display of the optotype or another additional stimulus to generate a modified stimulus at the same position, such as a version of the optotype that is transformed to simulate the application of a corrective lens. Some embodiments may then obtain a second feedback parameter associated with the modified stimulus. Such operations may be repeated for multiple transformed versions of the stimulus to help a user determine an optimal lens prescription for corrective lenses.

Some embodiments may transform content for various other types of applications. Some embodiments may generate transformation parameters to correct misalignments and then apply a transformation based on these transformation parameters to the presentation of an object in the context of a simulated environment being presented by a head-mounted device. For example, in the context of presenting a surgery simulation, some embodiments may apply transformation operations that would cause a −1.5 shift in the horizontal direction and a +0.5 shift in the vertical direction. If the simulation rendering a virtual surgical tool such that the viewer is supposed to be viewing the center of that virtual surgical tool at virtual coordinates [0, 0, 12], some embodiments may shift the center such that the center is present at the display positions [−1.5, 0.5, 12]. Such operations may enhance the visual acuity experienced by a user during the presentation of various types of content, such as content related to the exploration of a simulated environment, content related to videogames, content related to movies, etc.

The above-described embodiments of the present disclosure are presented for purposes of illustration and not of limitation, and the present disclosure is limited only by the claims which follow. Furthermore, it should be noted that the features and limitations described in any embodiment may be applied to one or more other embodiments herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods. Furthermore, not all operations of a flowchart need to be performed. In addition, the systems and methods described herein may be performed in real time. It should also be noted that the systems and/or methods described above may be applied to, or used in accordance with, other systems and/or methods.

Furthermore, the computing devices described in this disclosure may be any type of computing device unless otherwise stated, including, but not limited to, a laptop computer, a tablet computer, a hand-held computer, and/or other computing equipment (e.g., a server), including “smart,” wireless, wearable, and/or mobile devices. For example, while the client device 102 of FIG. 1 may be a head-mounted device, another type of mobile computing device may be possible. Furthermore, the embodiments described in this disclosure may include an individual device that performs some or all the operations described in this disclosure. Alternatively, other embodiments may include multiple computing devices acting collectively to perform some or all the operations described in this disclosure.

As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification, “a portion” refers to a part of, or the entirety (i.e., the entire portion), of a given item (e.g., data) unless the context clearly dictates otherwise. Furthermore, a “set” may refer to a singular form or a plural form, such that a “set of items” may refer to one item or a plurality of items.

In some embodiments, the operations described in this disclosure may be implemented in a set of processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on one or more non-transitory, machine-readable media (e.g., a set of machine-readable storage media), such as an electronic storage medium. Furthermore, the use of the term “media” may include a single medium or combination of multiple media, such as a first medium and a second medium. One or more non-transitory machine-readable media storing instructions may include instructions included on a single medium or instructions distributed across multiple media. For example, non-transitory media may act as one or more memory, where one or more memory may store program instructions that are written as source files or written in machine-executable program code. The processing devices may include one or more devices configured through hardware, firmware, and/or software to be specifically designed for the execution of one or more of the operations of the methods.

In some embodiments, the various computer systems and subsystems illustrated in FIG. 1 or FIG. 2 may include one or more computing devices that are programmed to perform the functions described herein. The computing devices may include one or more electronic storages (e.g., a set of databases accessible to one or more applications depicted in the system 100), one or more physical processors programmed with one or more computer program instructions, and/or other components. For example, the set of databases may include one or more relational databases. Alternatively, or additionally, the set of databases or other electronic storage used in this disclosure may include one or more non-relational databases.

The computing devices may include communication lines or ports to enable the exchange of information with a set of networks (e.g., a network used by the system 100) or other computing platforms via wired or wireless techniques. The network may include the internet, a mobile phone network, a mobile voice or data network (e.g., a 5G or Long-Term Evolution (LTE) network), a cable network, a public switched telephone network, or other types of communication networks or combination of communication networks. A network described by devices or systems described in this disclosure may include one or more communications paths, such as Ethernet, a satellite path, a fiber-optic path, a cable path, a path that supports internet communications (e.g., IPTV), free-space connections (e.g., for broadcast or other wireless signals), Wi-Fi, Bluetooth, near field communication, or any other suitable wired or wireless communications path or combination of such paths. The computing devices may include additional communication paths linking a plurality of hardware, software, and/or firmware components operating together. For example, the computing devices may be implemented by a cloud of computing platforms operating together as the computing devices.

Each of these devices described in this disclosure may also include electronic storages. The electronic storage may include one or more non-transitory machine-readable media (e.g., storage media) that electronically stored information. The storage media of the electronic storages may include one or both of (i) system storage that is provided integrally (e.g., substantially non-removable) with servers or client computing devices, or (ii) removable storage that is removably connectable to the servers or client computing devices via port (e.g., a USB port, a firewire port, etc.) or drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). An electronic storage may store software algorithms, information determined by the processors, information obtained from servers, information obtained from client computing devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent the processing functionality of a plurality of devices operating in coordination. The processors may be programmed to execute computer program instructions to perform functions described herein of subsystems described in this disclosure or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems described herein is for illustrative purposes, and is not intended to be limiting, as any of the subsystems described in this disclosure may provide more or less functionality than is described. For example, one or more of subsystems described in this disclosure may be eliminated, and some or all of its functionality may be provided by other ones of subsystems described in this disclosure. As another example, additional subsystems may be programmed to perform some, or all of the functionality attributed herein to one of the subsystems described in this disclosure.

With respect to the components of computing devices described in this disclosure, each of these devices may receive content and data via input/output (I/O) paths. Each of these devices may also include processors and/or control circuitry to send and receive commands, requests, and other suitable data using the I/O paths. The control circuitry may comprise any suitable processing, storage, and/or I/O circuitry. Further, some or all of the computing devices described in this disclosure may include a user input interface and/or user output interface (e.g., a display) for use in receiving and displaying data. In some embodiments, a display such as a touchscreen may also act as a user input interface. It should be noted that in some embodiments, one or more devices described in this disclosure may have neither user input interface nor displays and may instead receive and display content using another device (e.g., a dedicated display device such as a computer screen and/or a dedicated input device such as a remote control, mouse, voice input, etc.). Additionally, one or more of the devices described in this disclosure may run an application (or another suitable program) that performs one or more operations described in this disclosure.

Although the present invention has been described in detail for the purpose of illustration based on what is currently considered to be the most practical and preferred embodiments, it is to be understood that such detail is solely for that purpose and that the invention is not limited to the disclosed embodiments but, on the contrary, is intended to cover modifications and equivalent arrangements that are within the scope of the appended claims. For example, it is to be understood that the present invention contemplates that, to the extent possible, one or more features of any embodiment may be combined with one or more features of any other embodiment.

As used throughout this application, the word “may” is used in a permissive sense (i.e., meaning having the potential to), rather than a mandatory sense (i.e., meaning must). The words “include,” “including,” “includes,” and the like mean including, but not limited to. As used throughout this application, the singular forms “a,” “an,” and “the” include plural referents unless the context clearly indicates otherwise. Thus, for example, reference to “an element” or “the element” includes a combination of two or more elements, notwithstanding the use of other terms and phrases for one or more elements, such as “one or more.” The term “or” is non-exclusive (i.e., encompassing both “and” and “or”), unless the context clearly indicates otherwise. Terms describing conditional relationships (e.g., “in response to X, Y,” “upon X, Y,” “if X, Y,” “when X, Y,” and the like) encompass causal relationships in which the antecedent is a necessary causal condition, the antecedent is a sufficient causal condition, or the antecedent is a contributory causal condition of the consequent (e.g., “state X occurs upon condition Y obtaining” is generic to “X occurs solely upon Y” and “X occurs upon Y and Z”). Such conditional relationships are not limited to consequences that instantly follow the antecedent obtaining, as some consequences may be delayed, and in conditional statements, antecedents are connected to their consequents (e.g., the antecedent is relevant to the likelihood of the consequent occurring). Statements in which a plurality of attributes or functions are mapped to a plurality of objects (e.g., a set of processors performing steps/operations A, B, C, and D) encompass all such attributes or functions being mapped to all such objects and subsets of the attributes or functions being mapped to subsets of the attributes or functions (e.g., both/all processors each performing steps/operations A-D, and a case in which processor 1 performs step/operation A, processor 2 performs step/operation B and part of step/operation C, and processor 3 performs part of step/operation C and step/operation D), unless otherwise indicated. Further, unless otherwise indicated, statements that one value or action is “based on” another condition or value encompass both instances in which the condition or value is the sole factor and instances in which the condition or value is one factor among a plurality of factors.

Unless the context clearly indicates otherwise, statements that “each” instance of some collection has some property should not be read to exclude cases where some otherwise identical or similar members of a larger collection do not have the property (i.e., each does not necessarily mean each and every). Limitations as to the sequence of recited steps should not be read into the claims unless explicitly specified (e.g., with explicit language like “after performing X, performing Y”) in contrast to statements that might be improperly argued to imply sequence limitations (e.g., “performing X on items, performing Y on the X'ed items”) used for purposes of making claims more readable rather than specifying a sequence. Statements referring to “at least Z of A, B, and C,” and the like (e.g., “at least Z of A, B, or C”), refer to at least Z of the listed categories (A, B, and C) and do not require at least Z units in each category. Unless the context clearly indicates otherwise, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. Furthermore, unless indicated otherwise, updating an item may include generating the item or modifying an existing item. Thus, updating a record may include generating a record or modifying the value of an already-generated value in a record. Additionally, as used in the specification, “a portion” refers to a part of, or the entirety of (i.e., the entire portion), a given item (e.g., data) unless the context clearly dictates otherwise.

Unless the context clearly indicates otherwise, ordinal numbers used to denote an item do not define the item's position. For example, an item that may be a first item of a set of items even if the item is not the first item to have been added to the set of items or is otherwise indicated to be listed as the first item of an ordering of the set of items. Thus, for example, if a set of items is sorted in a sequence from “item 1,” “item 2,” and “item 3,” the first item of a set of items may be “item 2” unless otherwise stated.

### Enumerated Embodiments

The present techniques will be better understood with reference to the following enumerated clauses:
1. A method comprising: determining a prediction model output using a first prediction model based on feedback data stream obtained via a set of sensors of a head-mounted device; and in response to the prediction model output, generating an eye condition indication by providing, as a set of inputs to a second prediction model, the feedback data stream.
2. A method comprising: obtaining via a set of sensors of a head-mounted device, a feedback data stream; determining a prediction model output using a first prediction model based on the feedback data stream; and in response to the prediction model output, generating an eye condition indication by providing, as a set of inputs to a second prediction model, the feedback data stream.
3. A method comprising: presenting a first stimulus at a first display position on a head-mounted display of a head-mounted device; determining, with one or more sensors, a pupil center of a pupil directed at the first stimulus; determining a spatial displacement between the pupil center and a vertex of a headset lens of the head-mounted display; generating transformation parameters based on the spatial displacement; and presenting a second stimulus at a second display position by applying a transformation to an initial presentation position of the second stimulus based on the transformation parameters.
4. A method comprising: presenting a calibration stimulus at a first display position on a head-mounted display of a head-mounted device; determining, via one or more sensors, a pupil center of a pupil directed at the calibration stimulus; determining a spatial displacement between the pupil center and a vertex of a headset lens that is between the head-mounted display and the pupil; generating transformation parameters based on the spatial displacement between the pupil center and the vertex; applying a transformation to a second position based on the transformation parameters to determine a third position on the head-mounted display; and presenting a testing stimulus at the third position on the head-mounted display.
5. A method comprising: a headset lens, a head-mounted display, and an inward-facing sensor; and one or more processors and one or more memory storing program instructions that, when executed by the one or more processors, causes operations comprising: in connection with an initiation of a visual acuity test for a user, presenting a calibration stimulus at an optical infinity position on the head-mounted display; determining, with the inward-facing sensor, a pupil center of a pupil directed at the calibration stimulus, wherein the pupil is in a primary gaze position while directed at the calibration stimulus; determining a spatial displacement between the pupil center and a vertex of the headset lens; generating translational transformation parameters by generating a translation matrix based on the spatial displacement; and presenting a testing stimulus to the user during the visual acuity test while compensating for the spatial displacement by (1) applying a translational transformation to a testing stimulus position based on the translational transformation parameters to determine an adjusted testing stimulus position and (2) presenting the testing stimulus at the adjusted testing stimulus position on the head-mounted display.
6. The method of any of the embodiments above, wherein: determining the spatial displacement comprises determining a vertical displacement and a horizontal displacement; generating the transformation parameters comprises determining a set of translation parameters based on the vertical displacement and the horizontal displacement; and applying the transformation comprises applying a translation on the second position.
7. The method of any of the embodiments above, further comprising obtaining, from a data store, a parameter indicating a depth parameter, wherein generating the transformation parameters comprises generating the transformation parameters based on the depth parameter obtained from the data store.
8. The method of any of the embodiments above, wherein determining the spatial displacement further comprises determining a depth parameter based on measurements provided by a position sensor attached to a depth-controlling slider of the head-mounted device.
9. The method of any of the embodiments above, further comprising: detecting an orientation shift between the head-mounted device and a user's head; and determining a result indicating that the orientation shift satisfies a shift threshold, wherein presenting the calibration stimulus comprises presenting the calibration stimulus based on the result.
10. The method of any of the embodiments above, further comprising: presenting a second calibration stimulus at a second display position different from the first display position on the head-mounted display; determining, with the one or more sensors, a pupil orientation while the pupil directed at the second calibration stimulus; and determining a rotation angle based on the pupil orientation, wherein applying the transformation comprises (i) generating a rotation matrix based on the rotation angle and (ii) applying a rotation transformation to the second position.
11. The method of any of the embodiments above, wherein generating the transformation parameters comprises providing the spatial displacement to a machine learning model stored on a local memory of the head-mounted device.
12. The method of any of the embodiments above, further comprising presenting, at the third position, a pattern comprising a lighter color and a darker color.
13. The method of any of the embodiments above, further comprising performing a subjective refraction test by: presenting a third stimulus at the third position; obtaining a first feedback parameter associated with the third stimulus; modifying a display of the third stimulus to generate a modified stimulus at the third position; and obtaining a second feedback parameter associated with the modified stimulus.
14. The method of any of the embodiments above, wherein the calibration stimulus is positioned without overlapping with an optical infinity position of the head-mounted display.
15. The method of any of the embodiments above, further comprising: obtaining a device type identifier associated with the head-mounted device; and retrieving a position representing the vertex from a data store based on the device type identifier.
16. The method of any of the embodiments above, wherein the second stimulus comprises an optotype.
17. The method of any of the embodiments above, wherein: determining the spatial displacement comprises determining a vertical displacement and a horizontal displacement; generating the transformation parameters comprises determining a set of translation parameters based on the vertical displacement and the horizontal displacement; and applying the transformation comprises applying a translation on the initial presentation position.
18. The method of any of the embodiments above, further comprising: detecting an orientation shift between the head-mounted device and a user's head; and determining a result indicating that the orientation shift satisfies a shift threshold, wherein presenting the first stimulus comprises presenting the first stimulus based on the result.
19. The method of any of the embodiments above, wherein the first stimulus is a first calibration stimulus, the operations further comprising: presenting an other stimulus at an other display position different from the first display position on the head-mounted display; determining, with the one or more sensors, a pupil orientation while the pupil directed at the other stimulus; and determining a rotation angle based on the pupil orientation, wherein applying the transformation comprises (i) generating a rotation matrix based on the rotation angle and (ii) applying a rotation transformation to the initial presentation position.
20. The method of any of the embodiments above, wherein generating the transformation parameters comprises providing the spatial displacement to a machine learning model stored on a local memory of the head-mounted device.
21. The method of any of the embodiments above, wherein the first stimulus is positioned without overlapping with an optical infinity position of the head-mounted display.
22. The method of any of the embodiments above, further comprising performing a subjective refraction test by: presenting an additional stimulus at the second display position; obtaining a first feedback parameter associated with the additional stimulus; modifying a display of the additional stimulus to generate a modified stimulus at the second display position; and obtaining a second feedback parameter associated with the modified stimulus.
23. A tangible, non-transitory, machine-readable medium storing instructions that, when executed by a data processing apparatus, cause the data processing apparatus to perform operations comprising those of any of embodiments 1-22.
24. A system comprising one or more processors; and memory storing instructions that, when executed by the processors, cause the processors to effectuate operations comprising those of any of embodiments 1-22.
25. A system comprising means for performing any of embodiments 1-22.
26. A head-mounted device comprising: one or more inward-facing sensors and an inward-facing display; one or more processors and non-transitory media storing instructions that, when executed by the one or more of processors, cause the processors to effectuate operations comprising those of any of embodiments 1-22.

What is claimed is:

1. A head-mounted device for accurate visual acuity testing based on displacements between an eye and a lens vertex, the head-mounted device comprising:
a headset lens, a head-mounted display, and an inward-facing sensor; and
one or more processors and one or more memory storing program instructions that, when executed by the one or more processors, causes operations comprising:
in connection with an initiation of a visual acuity test for a user, presenting a calibration stimulus at an optical infinity position on the head-mounted display;
determining, with the inward-facing sensor, a pupil center of a pupil directed at the calibration stimulus, wherein the pupil is in a primary gaze position while directed at the calibration stimulus;
determining a spatial displacement between the pupil center and a vertex of the headset lens;
generating translational transformation parameters by generating a translation matrix based on the spatial displacement; and
presenting a testing stimulus to the user during the visual acuity test while compensating for the spatial displacement by (1) applying a translational transformation to a testing stimulus position based on the translational transformation parameters to determine an adjusted testing stimulus position and (2) presenting the testing stimulus at the adjusted testing stimulus position on the head-mounted display.

2. A method comprising:
presenting a calibration stimulus at a first display position on a head-mounted display of a head-mounted device;
determining, via one or more sensors, a pupil center of a pupil directed at the calibration stimulus;
determining a spatial displacement between the pupil center and a vertex of a headset lens that is between the head-mounted display and the pupil;
generating transformation parameters based on the spatial displacement between the pupil center and the vertex;
applying a transformation to a second position based on the transformation parameters to determine a third position on the head-mounted display; and
presenting a testing stimulus at the third position on the head-mounted display.

3. The method of claim 2, wherein:
determining the spatial displacement comprises determining a vertical displacement and a horizontal displacement;
generating the transformation parameters comprises determining a set of translation parameters based on the vertical displacement and the horizontal displacement; and
applying the transformation comprises applying a translation on the second position.

4. The method of claim 3, further comprising obtaining, from a data store, a parameter indicating a depth parameter, wherein generating the transformation parameters comprises generating the transformation parameters based on the depth parameter obtained from the data store.

5. The method of claim 3, wherein determining the spatial displacement further comprises determining a depth parameter based on measurements provided by a position sensor attached to a depth-controlling slider of the head-mounted device.

6. The method of claim 2, further comprising:
detecting an orientation shift between the head-mounted device and a user's head; and
determining a result indicating that the orientation shift satisfies a shift threshold, wherein presenting the calibration stimulus comprises presenting the calibration stimulus based on the result.

7. The method of claim 2, further comprising:
presenting a second calibration stimulus at a second display position different from the first display position on the head-mounted display;
determining, with the one or more sensors, a pupil orientation while the pupil directed at the second calibration stimulus; and
determining a rotation angle based on the pupil orientation, wherein applying the transformation comprises (i) generating a rotation matrix based on the rotation angle and (ii) applying a rotation transformation to the second position.

8. The method of claim 2, wherein generating the transformation parameters comprises providing the spatial displacement to a machine learning model stored on a local memory of the head-mounted device.

9. The method of claim 2, further comprising presenting, at the third position, a pattern comprising a lighter color and a darker color.

10. The method of claim 2, further comprising performing a subjective refraction test by:
presenting a third stimulus at the third position;
obtaining a first feedback parameter associated with the third stimulus;
modifying a display of the third stimulus to generate a modified stimulus at the third position; and
obtaining a second feedback parameter associated with the modified stimulus.

11. The method of claim 2, wherein the calibration stimulus is positioned without overlapping with an optical infinity position of the head-mounted display.

12. One or more non-transitory machine-readable media storing program instructions that, when executed by one or more processors, cause operations comprising:
presenting a first stimulus at a first display position on a head-mounted display of a head-mounted device;
determining, with one or more sensors, a pupil center of a pupil directed at the first stimulus;
determining a spatial displacement between the pupil center and a vertex of a headset lens of the head-mounted display;
generating transformation parameters based on the spatial displacement; and
presenting a second stimulus at a second display position by applying a transformation to an initial presentation position of the second stimulus based on the transformation parameters.

13. The one or more machine-readable media of claim 12, further comprising:
obtaining a device type identifier associated with the head-mounted device; and
retrieving a position representing the vertex from a data store based on the device type identifier.

14. The one or more machine-readable media of claim 12, wherein the second stimulus comprises an optotype.

15. The one or more machine-readable media of claim 12, wherein:
determining the spatial displacement comprises determining a vertical displacement and a horizontal displacement;
generating the transformation parameters comprises determining a set of translation parameters based on the vertical displacement and the horizontal displacement; and
applying the transformation comprises applying a translation on the initial presentation position.

16. The one or more machine-readable media of claim 12, further comprising:
detecting an orientation shift between the head-mounted device and a user's head; and
determining a result indicating that the orientation shift satisfies a shift threshold, wherein presenting the first stimulus comprises presenting the first stimulus based on the result.

17. The one or more machine-readable media of claim 12, wherein the first stimulus is a first calibration stimulus, the operations further comprising:
presenting an other stimulus at an other display position different from the first display position on the head-mounted display;
determining, with the one or more sensors, a pupil orientation while the pupil directed at the other stimulus; and
determining a rotation angle based on the pupil orientation, wherein applying the transformation comprises (i) generating a rotation matrix based on the rotation angle and (ii) applying a rotation transformation to the initial presentation position.

18. The one or more machine-readable media of claim 12, wherein generating the transformation parameters comprises providing the spatial displacement to a machine learning model stored on a local memory of the head-mounted device.

19. The one or more machine-readable media of claim 12, wherein the first stimulus is positioned without overlapping with an optical infinity position of the head-mounted display.

20. The one or more machine-readable media of claim 12, further comprising performing a subjective refraction test by:
presenting an additional stimulus at the second display position;
obtaining a first feedback parameter associated with the additional stimulus;
modifying a display of the additional stimulus to generate a modified stimulus at the second display position; and
obtaining a second feedback parameter associated with the modified stimulus.
